(19) United States
(12) Patent Application Publication
(10) Pub. No.: US 2015/0173667 A1
(43) Pub. Date: Jun. 25, 2015

(54) SYSTEM AND METHOD OF PRESSURE MAPPING AND 3-D SUBJECT REPOSITIONING FOR PREVENTING PRESSURE WOUNDS

(71) Applicant: Enhanced Surface Dynamics, Inc., Palo Alto, CA (US)

(72) Inventors: Amir Ben Shalom, Modiin (IL); Efrat Zaritzky, Tel Aviv (IL); Kristen Thurman, Tempe, AZ (US); Lior Greenstein, Tel Aviv (IL)

(21) Appl. No.: 14/419,761

(22) PCT Filed: Jul. 31, 2013
(86) PCT No.: PCT/IB13/56287

§ 371 Date: Feb. 5, 2015

Related U.S. Application Data
(60) Provisional application No. 61/679,826, filed on Aug. 6, 2012.
(60) Provisional application No. 61/716,781, filed on Oct. 22, 2012.

(51) Int. Cl. A61B 5/00; G06F 19/00; A61B 5/11; G01L 1/14; A61G 7/057; A61G 5/10; G08B 21/04; G01L 1/26

(52) U.S. Cl. CPC A61B 5/447; G08B 21/0461; G08B 21/0446; G06F 19/3431; G01L 1/26; G01L 1/14; A61G 7/057; A61G 5/10; A61B 5/486; A61B 5/7275; A61B 5/7278; A61B 5/746; A61B 5/743; A61B 5/1116; A61B 5/1121; A61G 2203/20; A61G 2203/34; A61G 2203/42; A61B 2560/0475; A61B 2562/0247; A61B 2562/0219

(57) ABSTRACT

A pressure wound prevention system operable to prompt at least one caregiver to reposition a subject at risk of developing a pressure wound. The system includes a pressure-detection surface comprising a plurality of pressure sensors configured to monitor pressure exerted upon the pressure-detection surface by the subject as well as orientation sensors configured to monitor an angle of the pressure-detection surface. A processor is operable to interpret and analyze data from the sensors and to provide indications to a caregiver when the subject requires repositioning.

### FIELD OF THE DISCLOSURE

The disclosure herein relates to a pressure monitoring system and method for preventing the development of pressure wounds. In particular the disclosure relates to pressure distribution mapping and its use in recommending weight redistribution of a subject.

### BACKGROUND

Pressure wounds, e.g. decubitus ulcers, which are commonly known as pressure ulcers or bedsores, are lesions developed when a localized area of soft tissue is compressed between a bony prominence and an external surface for a prolonged period of time. Pressure ulcers may appear in various parts of the body, and their development is affected by a combination of factors such as unrelieved pressure, friction, shearing forces, humidity and temperature.

Currently, about 10%-15% of hospitalized patients are estimated to have bedsores at any one time (Source: Medicare website 2009). Although easily preventable and treatable if found early, bedsores are painful, and treatment is both difficult and expensive. In many cases, bedsores can prove fatal—even under the auspices of medical care.

An effective way of dealing with pressure wounds is to prevent them. Existing preventive solutions are either passive (e.g., various types of cushioning) or active, including a range of dynamic mattresses that alternate the inflation/deflation of air cells. However, dynamic mattresses have a tendency to redistribute pressure from unnecessary locations thereby needlessly creating higher pressure in the sensitive areas, which may not effectively prevent pressure wounds. Moreover, such mattresses are typically designed for patients lying down in hospital beds, and hardly answer the needs of individuals who spend considerable amounts of time sitting up, confined to a wheelchair or the like.

A common preventive approach is keeping a strict routine of turning the patient every 2-3 hours or other determined time. This is a difficult, labor intensive and costly task and in many cases may be performed too late.

There is, therefore, a need for a reliable, cost effective system and method for pressure distribution mapping and monitoring as well as providing notification of the need for repositioning and the recommendation for the appropriate orientation preventing the development of pressure wounds, such as decubitus ulcers or bedsores, and providing relief to the patient. The present disclosure addresses this need.

### SUMMARY

It is an advantage of the current disclosure that it may improve the treatment of patients suffering from bedsores or at risk of developing pressure wounds. An alert may be sent to a caregiver associated with indications for repositioning, and aimed at reducing the possibility of developing pressure wounds. In particular, the system disclosed herein may enable the changing of the orientation of pressure adjustable detection surface sections, where each section may be tilted at a different angle. Furthermore, the system described herein may provide a more reliable recommendation plan to reduce bedsore development and may also serve for pressure mapping and monitoring of surfaces attached to a wheelchair, for example.

Aspects of the disclosure present a system for preventing pressure wounds, operable to prompt at least one caregiver to reposition a subject at risk of developing a pressure wound, the system comprising: at least one pressure-detection surface comprising a plurality of pressure sensors configured to monitor pressure exerted upon the subject, at least one processor operable to interpret and analyze data from the sensors, at least one notification mechanism in communication with at least one processor, the notification mechanism operable to initiate at least one repositioning alert and at least one display unit configured to present the repositioning alert.

The pressure wound prevention system may use different types of pressure-detection surfaces. Such detection surfaces may be selected from a group consisting of a mattress, bed, chair, stool, sofa, wheelchair, rocking chair, coverlet and bench. Furthermore, the pressure wound prevention system may further comprise at least one memory unit, configured to store the data from the sensors and the resulting calculated data by the system's processor.

It is a particular feature of the disclosure that the pressure-detection surface further comprises at least one orientation sensor configured to monitor an angle of the pressure-detection surface.

Optionally or additionally, the pressure-detection surface of the pressure wound prevention system may comprise a plurality of adjustable sections.

Optionally, the pressure wound prevention system, wherein referenced, may include an orientation sensor that may comprise an accelerometer. In one embodiment, such an accelerometer may be a three dimensional accelerometer.

The pressure wound prevention system may use a method for processing sensor data measurements and is configured to detect pressure and orientation for each section of the adjustable pressure-detection surface. The method comprises using data, for each surface section, from a plurality of pressure sensors configured to detect pressure distribution and at least one orientation sensor configured to detect angle indication.

The method may initially identify the surface sections of the adjustable pressure-detection surface, and thereafter receive pressure data from the pressure sensors for each surface section, and receive orientation data from the orientation sensors for each surface section, perform a data analysis process, determine a notification indication for the caregiver and present such data on the display units.

Accordingly, in order to perform the data analysis, filtering random movement of the orientation sensors may be performed for each surface section, and the perpendicular and tangential forces and the shear stress of the subject over each surface section of the pressure-detection surface may be calculated. Thereafter, the risk level of the subject developing pressure wounds may be determined, providing an output based upon calculated data, and may be configured to send an alert to a caregiver indicating a repositioning requirement.

It is noted that when referencing output data, the method may provide the ability of displaying a current orientation indication and a recommended orientation indication for each section on at least one display unit, local or centrally managed.

It is further noted that when referencing output data, the method may provide the ability of displaying a pressure distribution map for each section on the at least one display unit.

Optionally or additionally, when referencing orientation indication, the method may refer to a three dimensional reference, indicating lateral tilt values.

Optionally or additionally, the method may further comprise the ability of storing the data in at least one data storage unit.

Another aspect of the disclosure presents a system associated with a seated subject on a wheelchair, for preventing pressure wounds, operable to prompt at least one caregiver to reposition a subject at risk of developing a pressure wound, the system comprising: at least one pressure-detection surface comprising a plurality of pressure sensors configured to monitor pressure exerted upon the surface possibly by the subject, at least one processor operable to interpret and analyze data from the sensors, at least one data storage unit operable to store the data from said sensors and said processor, at least one notification mechanism in communication with at least one processor, the notification mechanism operable to initiate at least one repositioning alert, a power supply unit operable to provide operational power, a data recording manager in communication with the at least one data storage unit and the at least one processor and operable to manage data collection, a communication unit operable to provide access to the data; and at least one display unit configured to present the repositioning alert.

Optionally or additionally, the pressure-detection surface of the pressure wound prevention system for a seated subject may comprise a plurality of adjustable sections.

It is noted that the data recording manager may comprise at least one memory unit configured to store the data from the sensors and the processor according to a recordation schedule, for example at regular time intervals.

The display unit is operable to display the relevant screens to allow configuring the settings of the system, display collected or analyzed data, trigger alerts, present indications to the caregiver and the like.

It is noted that when referencing output data, the notification mechanism is operable to initiate a display of a series of pressure distribution maps representing the data sequentially. Additionally, the notification mechanism is also operable to generate a recommendation plan of a weight shift schedule, possibly for each of the surface sections.

It is further noted that when referencing output data, the notification mechanism is operable to generate a daily target achievement plan.

Optionally, the pressure wound prevention system for a seated subject, wherein referenced, may include an orientation sensor that may comprise an accelerometer, configured to monitor an angle of the pressure-detection surface. In one embodiment, such an accelerometer may be a three dimensional accelerometer.

The pressure wound prevention system for a seated subject, say in a wheelchair, may use a method for processing sensor data measurements and may be configured to detect pressure from at least one pressure-detection surface comprising a plurality of pressure sensors configured to detect pressure distribution.

The method may initially identify the surface sections of the adjustable pressure-detection surface, and thereafter receive pressure data from the pressure sensors for each surface section, perform a data analysis process, storing data records in at least one data storage unit according to a recordation schedule, say at regular time intervals, determine a notification indication for the caregiver and present such data on the at least one display unit.

Accordingly, in order to perform the data analysis, the method may include calculating vertical forces and shear forces of the seated subject over each of the surface sections of the pressure-detection surface, determining a risk of a seated subject developing pressure wounds, providing an output, which may be based upon calculated data, determined from a look-up table or otherwise decided upon, and sending an alert to a caregiver indicating a repositioning requirement.

It is noted that when referencing output data, the method may provide the ability of displaying on a display unit a pressure distribution map representing said data sequentially at regular time intervals possibly for each of the sections.

It is further noted that when referencing output data, the method may provide the ability of displaying on a display unit a recommendation plan of a weight shift schedule possibly for each of the sections.

Additionally or optionally, when referencing output data, the method may generate a daily target achievement plan displayed on at least one display unit.

It is noted that the system may comprise at least one orientation sensor configured to detect angle indication, and the method may provide the ability of displaying a current orientation indication and a recommended orientation indication for each section on at least one display unit. Accordingly, optionally or additionally, the method may further comprise the step of receiving orientation data from the orientation sensor for each surface section after the step of receiving pressure data.

Optionally or additionally, when referencing orientation indication, the method may refer to a three dimensional reference, indicating lateral tilt values.

It is noted that in order to implement the methods or systems of the disclosure, various tasks may be performed or completed manually, automatically, or combinations thereof. Moreover, according to selected instrumentation and equipment of particular embodiments of the methods or systems of the disclosure, some tasks may be implemented by hardware, software, firmware or combinations thereof using an operating system. For example, hardware may be implemented as a chip or a circuit such as an ASIC, integrated circuit or the like. As for software, selected tasks according to embodiments of the disclosure may be implemented as a plurality of software instructions being executed by a computing device using any suitable operating system.

In various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally or alternatively, the data processor may access a non-volatile storage, for example, a magnetic hard-disk, flash-drive, removable media or the like, for storing instructions and/or data. Optionally, a network connection may additionally or alternatively be provided. User interface devices may be provided such as visual displays, audio output devices, tactile outputs and the like. Furthermore, as required, user input devices may be provided such as keyboards, cameras, microphones, accelerometers, motion detectors or pointing devices such as mice, roller balls, touch pads, touch sensitive screens or the like.

### BRIEF DESCRIPTION OF THE FIGURES

For a better understanding of the embodiments and to show how they may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1A is a schematic block diagram of the main components of a pressure monitoring system according to an embodiment;

FIG. 1B is a schematic of an extended pressure monitoring system including a plurality of sub-systems of different kinds;

FIG. 2 depicts an isometric projection of a sample embodiment of a pressure-detection surface;

FIG. 3A shows a possible display screen for indicating the current orientation and pressure distribution of subject's body on a three section adjustable surface;

FIG. 3B shows a possible display screen for indicating a recommendation orientation plan of subject's body on a three section adjustable surface;

FIG. 3C shows a possible combined display screen of a recommendation plan on a 24 hour schedule, while indicating the orientation and pressure distribution for three sections of the subject's body;

FIGS. 4A-D are representations of how pressure data may be displayed on a screen of a display system according to an embodiment;

FIG. 5A is a schematic force diagram representing forces acting upon a subject in a tilted section of an adjustable surface;

FIG. 5B is a schematic of a three dimensional axis configuration showing possible rotation directions;

FIG. 6A is a flowchart representing possible selected actions of a general method for performing pressure and orientation data analysis;

FIG. 6B is a flowchart representing possible selected actions of a method for providing an orientation-based indication to a caregiver;

FIG. 6C is a flowchart representing possible selected actions of a method for performing data analysis for an orientation-based indication to a caregiver;

FIG. 7A is a block diagram showing selected elements of a hardware system for use in pressure mapping system possibly for integration onto a wheelchair or the like;

FIG. 7B is an illustration representing an example of a wheelchair based embodiment configured for using a pressure-detection monitoring system;

FIG. 7C is an illustration representing a monitor and a mounting system on a sample wheelchair embodiment using a pressure-detection monitoring system;

FIGS. 8A-C are representations of possible screenshots for a pressure map display, and selected system settings display screens of an example of a wheelchair based embodiment;

FIGS. 9A-E are representations of weight shift notification and performance processes for a caregiver handling a subject on a wheelchair based embodiment;

FIGS. 10A-B are representations of a daily target achievement system and data tab selection for a caregiver handling a subject on a wheelchair based embodiment; and

FIGS. 11A-B are representations of possible data analysis reporting such as snapshots or video streaming for a caregiver handling a subject on a wheelchair based embodiment.

### DETAILED DESCRIPTION

Aspects of the present disclosure relate to a pressure and orientation monitoring system and method directed towards preventing pressure wounds, such as decubitus ulcers or bedsores, from developing in a subject supported for example in a lying or in a seated position.

Embodiments described herein disclose a pressure-detection monitoring system, such as for preventing the development of pressure wounds. The pressure-detection monitoring system may be configured to detect pressure created by a subject over a pressure-detection surface and to monitor surface orientations. Bedsores may develop when tissue, say a bony prominence, is in contact with an external surface for a prolonged period of time. Bedsores may also develop when tissue is forced in a direction parallel to the surface due to friction and shear forces resulting from patient movements, a nurse moving the patient, or bed or wheelchair movements.

### Pressure Detection—Functional Components:

The pressure-detection monitoring system of the disclosure may include at least one pressure-detection surface, a plurality of sensors configured to detect pressure, at least one orientation sensor configured to detect surface angles, at least one driving unit configured to supply electrical potential to the sensors, at least one control unit configured to control the driving units and receive data from the sensors, at least one processor configured to interpret and analyze the data and at least one display configured to present the data. The system may further include at least one storage unit configured to store data from the control units and processors.

The number of pressure-detection sections of a surface may vary according to need. Pressure-detection surfaces are typically integrated into areas of a bed or a sitting apparatus, such as a wheelchair, for example, which are designed to hold body parts that are prone to develop pressure wounds.

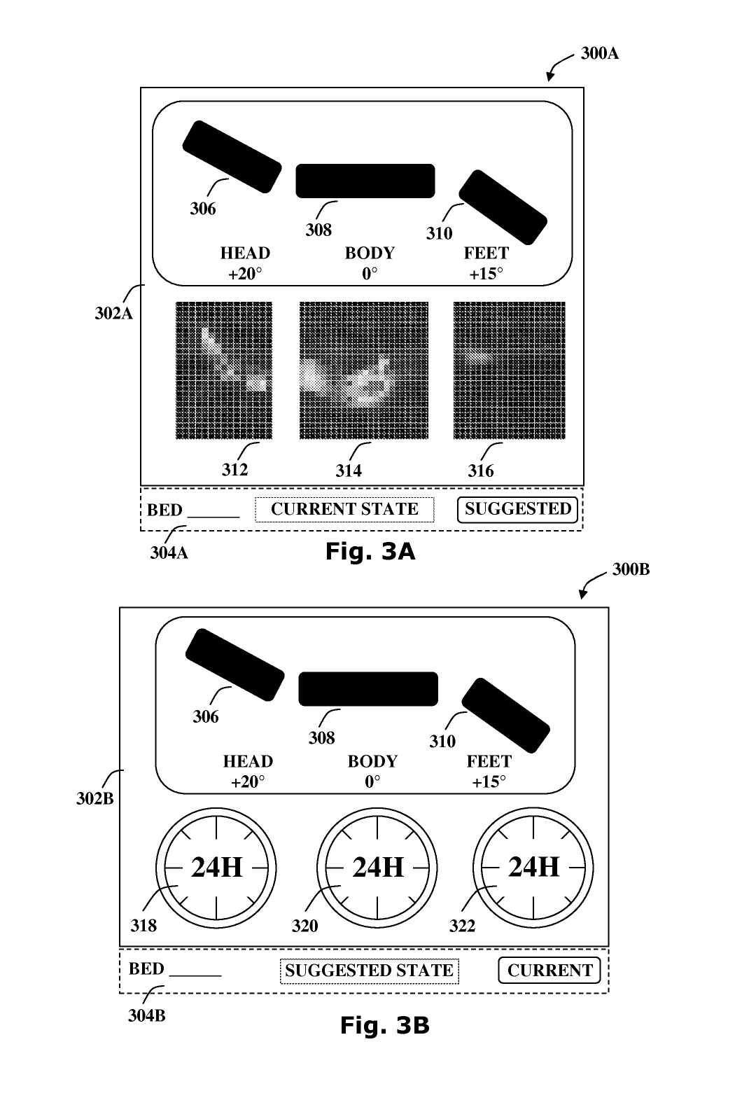

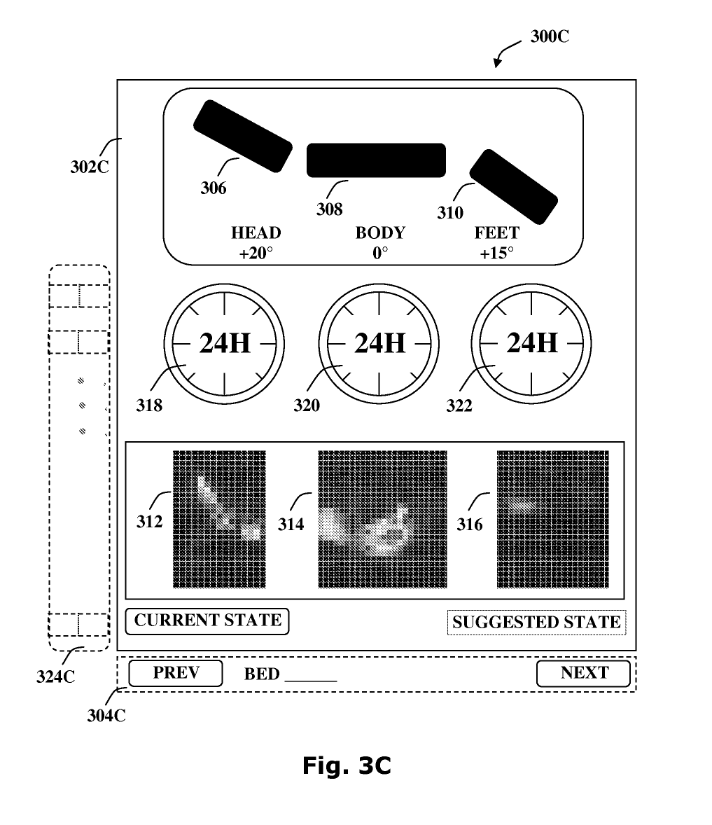

The display screen of the pressure-detection monitoring system may identify different body sections, such as head, body, and feet sections of the pressure-detection surface, for example, such as shown in the illustrative display of FIGS. 3A-C. The identification of the sections may allow pressure distribution to be more easily determined when a subject is in an unusual body position.

This arrangement may be useful, for example, when a subject is resting upon an adjustable bed including a plurality of sections such as is known in the art. By way of illustration, a hospital bed may be constructed from three or more sections which may each be configurable such that the patient adopts various postures. The pressure distribution over the sections may be recorded and displayed, say, on a display showing the pressure distribution map for each section of the bed.

Additionally or alternatively, the pressure-detection system components may be integrated into a wheelchair. Embodiments of the pressure-detection surface may provide a pressure sensing cover to be secured to a wheelchair cushion and its back seat to allow caregivers and patients to have real-time pressure distribution monitoring accessible on an individual handheld device to identify high risk pressure areas and off-load them accordingly. Optionally, pressure-detection surface sections may be further be integrated into the arm rests and the foot rests.

### Pressure Detection—Orientation Sensing Functionality:

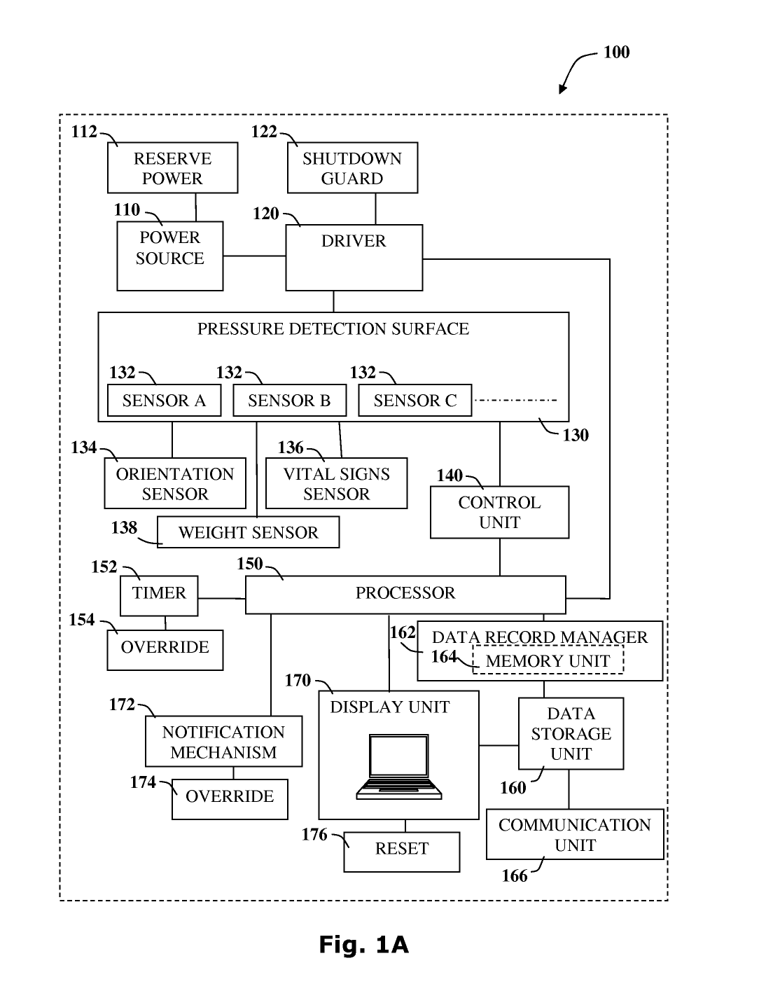

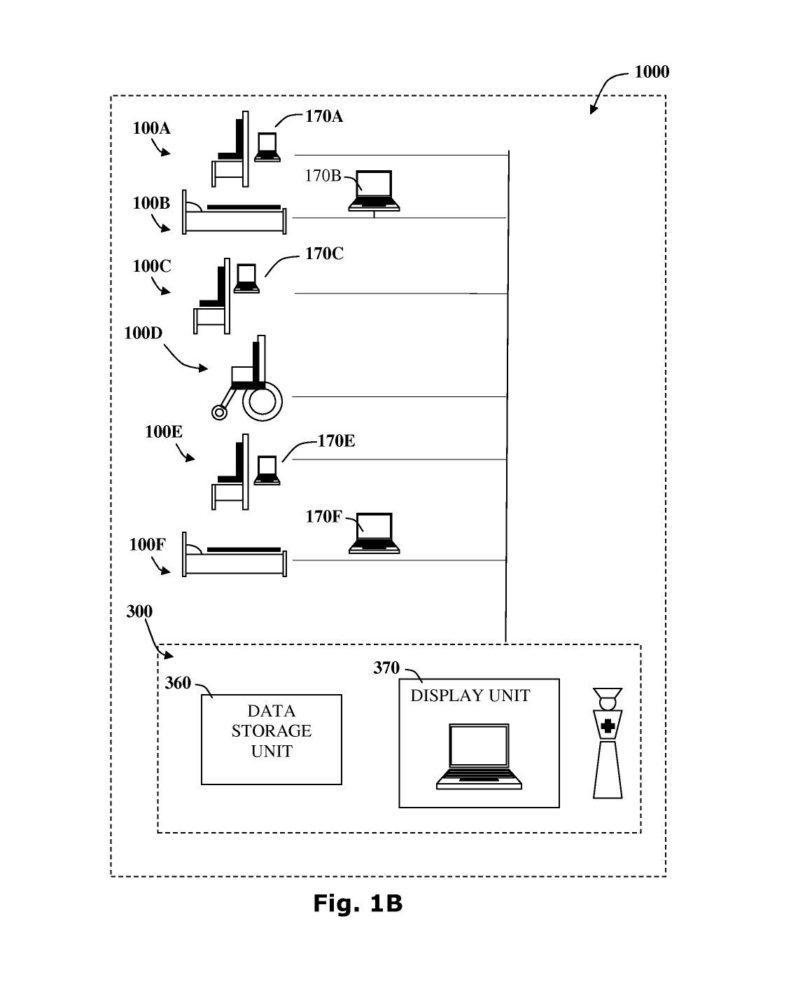

It is a particular feature of the current disclosure that orientation sensors 134 (FIG. 1A) may be incorporated into sections of the pressure-detection surface, and may determine the angle of each section. Accordingly, the orientation sensors 134 may serve to determine the patient's posture orientation which may be displayed to a caregiver. Furthermore, the risk of developing pressure wounds may be more accurately determined, alerting the caregiver when the risk level exceeds a threshold, for example, and providing a scheduled repositioning plan or the like.

Various orientation sensors may be used for the purpose of monitoring angles as described herein, for example, a plurality of accelerometers may be incorporated into a pressure-detection surface such as an overlay, a coverlet, a mattress, a wheelchair, a bed or the like to monitor the angle of the section to the horizontal. Where appropriate, a three dimensional accelerometer may be used to provide information relating to the angle of a section around a plurality of axes, say, lateral as well as longitudinal tilt of the surface.

An accelerometer, as referred to hereinafter, is an electromechanical device operable to measure acceleration forces. These forces may be static, like the constant force of gravity pulling, or they could be dynamic, caused by moving or vibrating the accelerometer. By measuring the amount of static acceleration due to gravity, the angle at which the device is tilted with respect to the ground may be determined. By sensing the amount of dynamic acceleration, one may analyze the way the sensor is moving.

Accordingly, the display may provide an indication of the orientations of each monitored section of the subject's body. For example, a user interface may indicate that the head portion is orientated at an angle of +20 degrees to the horizontal, the body portion is orientated at an angle of 0 degrees to the horizontal, and the lower limbs portion is orientated at an angle of +15 degrees to the horizontal. Such an indication may be presented as a text string, as a map or by way of an icon such as shown in the illustrative display of FIG. 3A-C, or as may otherwise occur to those skilled in the art.

It is particularly noted that the information regarding orientation and tilt of a subject's body section may be useful in calculating the risk of a subject developing pressure wounds as a result of shear forces.

Furthermore, the display may additionally provide a notification or recommendation that a caregiver should reposition the subject in three dimensions, by changing the orientation of the support itself or as an alternative by changing the two dimensional position of the subject upon the surface. Accordingly, the display may be used to provide guidance to a caregiver for repositioning a patient, answering the patient's particular needs.

It is noted that the systems and methods of the disclosure herein may not be limited in their application to the details of construction and the arrangement of the components or methods set forth in the description or illustrated in the drawings and examples. The systems and methods of the disclosure may be capable of other embodiments or of being practiced or carried out in various ways.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting.

### Embodiments of Multi-surface Repositioning System:

Reference is now made to the block diagram of FIG. 1A showing an embodiment of a pressure monitoring system 100. The system may include at least one pressure-detection surface 130 including a plurality of pressure sensors 132 and at least one orientation sensor 134 for each adjustable surface section, a driver 120, a control unit or controller 140 which may be connected to a power source 110, a processor 150, data storage 160 and a display system 170. Power may be supplied via a power cord connected to a wall outlet, or via battery power, optionally rechargeable. Battery support also allows for movement of the bed without requiring a powering off of the system. As a safety measure and for compliance tracking, caregiver authentication may be required via a shutdown guard 122 to confirm powering off the control unit 140, such as with entry of a caregiver's employee identification number.

In one embodiment, the driver 120 selectively supplies voltage to sensors 132 in the pressure-detection sheet, the processor 150 monitors the potential across the sensors 132, calculates impedance values for each sensor 132, and stores that data in data storage 160. The stored data may be further processed, analyzed, and displayed on a display system 170, such as computer screens, laptops, PDAs, cellular phone screens, printed sheets, integrated LCD screens (e.g. TFT, touch screen) and the like. Although presented in the block diagram of FIG. 1A as separate blocks, the system may optionally be integrated into a stand-alone system.

It is further noted that the data storage unit 160 be managed by a data recording manager 162 operable to record data, possibly in a memory unit 164, according to a schedule, periodically, manually or the like as required. Additionally or alternatively, data in the data storage unit 160 may be accessed via a communication unit 166, such as a wireless data transmitter, for example via radio waves using protocols such as WIFI, BLUETOOTH, ZIGBEE or the like.

Referring now to FIG. 1B, shown is an extended pressure wound prevention system 1000 including a plurality of pressure wound prevention sub-systems 100A-F in communication with a common remote control center 300. The pressure wound prevention sub-systems 100A-F may monitor various subjects in various positions for example on beds 100B, 100F, chairs 100A, 100C, 100E and wheelchairs 100D in a hospital, care home or the like, and may be configured to communicate with a remote control center 300, for example at a nursing station via a data communication line. It will be appreciated that in embodiments where the pressure-detection surface is configured to move, such as where the subject is seated in a wheelchair or the like, wired data cables may be inappropriate and data transmission via wireless means may be preferred, for example via radio waves using protocols such as WIFI, BLUETOOTH, ZIGBEE or the like.

Alternatively, the plurality of pressure wound prevention sub-systems 100A-F may be located remotely from one another, for example each in an individual home, and the remote control center 300 may be a manned monitoring station. In such systems, a data communication line may be provided via a cellular network, connections to the internet or the like.

It is further noted that a single pressure wound prevention system may include multiple pressure-detection mats, for example and without limitation, two mats located on a seat of a chair and on a back of a chair.

The remote control center 300 typically includes data storage 360 for storing data from the sub-systems 100A-F and a display 370 for presenting the data as required. It will be appreciated that the control center 300 may additionally provide processing and driving functionality for controlling multiple sub-systems. Optionally each pressure wound prevention sub-system 100A-F may have its own dedicated display 170 (see FIG. 1A) for processing, storing and displaying data locally.

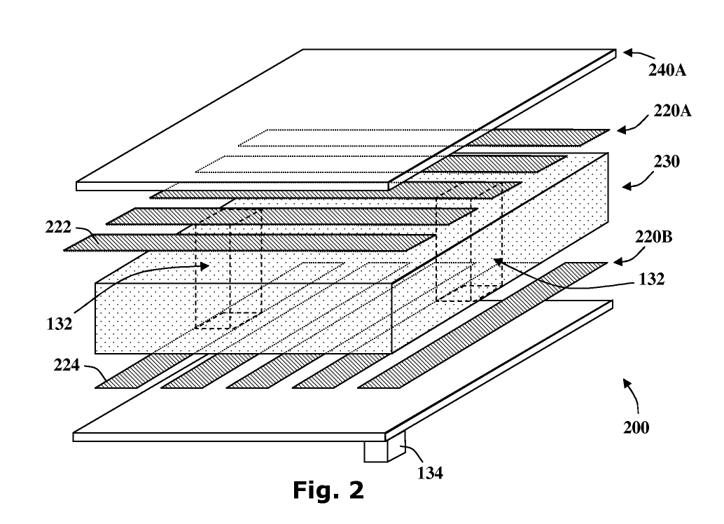

Reference is now made to FIG. 2 showing an embodiment of a pressure-detection surface 200 comprising a plurality of pressure sensors 132 arranged in a form of a matrix and at least one orientation sensor 134. The surface may have two layers 220A, 220B of conductive material separated by an insulating layer 230 of isolating material. Each of the conductive layers 220A, 220B may include parallel conductive strips 222, 224 and the two conductive layers 220A, 220B of strips 222, 224 may be arranged orthogonally such that in one conductive layer 220A the strips 222 are horizontal and in the other conductive layer 220B the strips 224 are vertical. Horizontal and vertical are used herein to describe the relative relationship of strips 222, 224 to one another, and these terms are not intended to be otherwise limiting. Each strip 222, 224 may be wired to a control unit and may operate under a low voltage source.

A capacitance sensor 132, such as that incorporated in pressure-detection surface 200, may be based on the capacitance between the sections of the conducting strips 222, 224 overlapping at each “intersection” of a vertical conductive strip 224 with a horizontal conductive strip 222. These capacitance sensors are configured such that pressing anywhere on their surface changes the spacing between the two conductive layers 220A, 220B, and consequently the capacitance of the intersection. A driver (not shown) may selectively provide electric potential to the vertical strip 224 and the electrical potential may be monitored on the horizontal strip 222 such that the capacitance of the overlapping section may be determined.

It may be noted that by providing an oscillating electric potential across each pressure sensor 132 and monitoring the alternating current produced thereby, the impedance of the intersection may be calculated and the capacitance of the intersection determined. Thus, where the mechanical properties of the sensor 132 are known, the pressure applied upon the sensor 132 may be deduced.

It may further be noted that the pressure-detection surface 200 may comprise a plurality of adjustable sections and at least one orientation sensor 134 for each adjustable section of the pressure-detection surface 200.

The pressure-detection surface 200 may further include additional sensors (not shown) configured to monitor additional factors, particularly those influencing the development of bedsores, such as temperature, humidity, or the like. Such additional sensors may be configured to monitor the factors continuously or intermittently as appropriate to detect high risk combinations of factors. Such measurements may be recorded and stored in data storage such as a database for further analysis.

Optionally, the pressure-detection surface 200 may be placed underneath or otherwise integrated with other material layers such as used in standard bed sheets. The additional materials may confer further properties as needed for a particular application. The conductive material of the sensors 132 may be wrapped by an isolating, water resistant, breathable cover sheet or the like, allowing minimum discomfort to the subject resting on the sheet.

In some embodiments, the materials are selected such that the conductive layers 220A, 220B and insulating layers 230 are flexible. The insulation material may be a compressible sponge-like, airy or porous material (e.g., foam), allowing for a change in density when pressure is applied to it.

Reference is now made to FIGS. 3A-C showing possible visual presentations of a subject's current state while resting on a pressure-detection surface, and suggested orientation changes with the possible ability of toggling between the two presentation states. The presentation may represent subject pressure distribution, the orientations of the separate surface sections, as well as other factors which may be useful for avoiding development of pressure wounds.

It is noted that the display configurations shown in FIGS. 3A-C may apply to a screen display 170 (FIG. 1A) monitoring a sub-system 100 A-H (FIG. 1B). Optionally or alternatively, the display configuration may apply to a central screen display, such as the display unit 370 (FIG. 1B) that may be used for a centrally monitoring pressure-detection system.

It is further noted that the display configurations detailed herein are described by way of example and may not be limited to the arrangement of the components or methods set forth in the description or illustrated in the drawings and examples. The various displays and methods of the disclosure may be capable of other embodiments or of being practiced or carried out in various ways.

Alternative methods and display configurations similar or equivalent to those described herein may be used in the practice or testing of embodiments of the disclosure. Nevertheless, particular methods and materials are described herein for illustrative purposes only. The materials, methods, and examples are not intended to be necessarily limiting.

Reference is now made to FIG. 3A showing a representation of a screen display 300A of a possible display for indicating the current orientation and pressure distribution of three sections of subject's body—head, body and feet, for example. This may be applicable, for example, where the subject is lying over three sections of an adjustable pressure distribution surface, such as a three-sectioned bed in a hospital, or three-sectioned wheelchair. It is further noted that various supporting platforms may have more or less sections. For example, a wheelchair or recliner, for example, may have multiple sensing sections for monitoring pressure against arm rests, back rests, head rests and the like.

The screen display 300A may include a display frame 302A with an upper viewing frame displaying a current orientation for each section of the adjustable pressure distribution surface 306, 308 and 310, and a lower viewing frame displaying the pressure distribution map for each section of the adjustable pressure distribution surface 312, 314 and 316.

Accordingly, the upper viewing frame of the screen display 300A may provide an indication of the orientations of each monitored section of the subject's body. For example, a user interface may indicate that the head portion is orientated at an angle of +20 degrees to the horizontal, the body portion is orientated at an angle of 0 degrees to the horizontal, and the lower limbs or feet portion is orientated at an angle of +15 degrees to the horizontal. Such an indication may be presented as a text string or may be displayed graphically as a map or an icon or as may otherwise occur to those skilled in the art.

Optionally or additionally, the screen display 300A may include a control panel 304A, displaying associated information such as bed number and state indicator, using ‘CURRENT’, for example, indicating the display is of current readings (as illustrated in FIG. 3A) or ‘SUGGESTED’ indicating the display is of a recommended plan for repositioning or re-orienting (as illustrated in FIG. 3B). Optionally or additionally, the control panel may include a toggling button, enabling one to switch between the display of ‘CURRENT’ readings (FIG. 3A) to ‘SUGGESTED’ readings (FIG. 3B) and vice versa.

Optionally or additionally, the lower viewing frame of the screen display 300A may include a display showing the pressure distribution map for each section of the adjustable pressure distribution surface, such as sections supporting head 312, body 314 and feet 316. Other sections may be additionally or alternatively represented as required.

Alternatively, the lower viewing frame may include only a toggle button for switching between the displays of ‘CURRENT’ readings (FIG. 3A) and ‘SUGGESTED’ readings (FIG. 3B).

It may be noted that the current display may be continually updated with the changes of actual orientation values for each surface section, or may be updated at a configurable time interval.

Reference is now made to FIG. 3B showing a representation of screen display 300B of a possible display for indicating the suggested plan for re-orientation of the sections, such as the head section, body section and feet section, for example, of the adjustable pressure distribution surface, such as a bed in a hospital.

The screen display 300B may include a display frame 302B with an upper viewing frame displaying the orientation for each section of the adjustable pressure distribution surface 306, 308 and 310 and a lower viewing frame displaying a recommendation plan over a 24 hour period for each surface section, as represented by a clock. This representation may enable a user to view all recommendations, or the recommendation at a specified time period.

Accordingly, the upper viewing frame of the screen display 300B may provide an indication of the orientations of each monitored section of the subject's body. For example, a user interface may indicate that the head portion is orientated at an angle of +20 degrees to the horizontal, the body portion is orientated at an angle of 0 degrees to the horizontal, and the lower limbs or feet portion is orientated at an angle of +15 degrees to the horizontal. Such an indication may be presented as a text string or may be displayed graphically as a map or an icon or as may otherwise occur to those skilled in the art.

Optionally or additionally, the screen display 300B may include a control panel 304B, displaying associated information such as the bed number and state indicator, using ‘CURRENT’, for example, indicating the display is of current readings (as illustrated in FIG. 3A) or ‘SUGGESTED’ indicating the display is of a recommended plan for repositioning or re-orienting (as illustrated in FIG. 3B). Optionally or additionally, the control panel may include a toggling button, enabling one to switch between the display of ‘CURRENT’ readings (FIG. 3A) to ‘SUGGESTED’ readings (FIG. 3B) and vice versa.

Additionally, the lower viewing frame of the screen display 300B may include a view enabling selection and display of the recommended plan for each section of the adjustable pressure-detection surface, such as sections supporting the head 318, body 320 and feet 322. Such a recommendation plan may simply suggest a specific re-orientation or provide for a scheduled set of recommendations, such as a 24 hour cycle, for example.

Alternatively, the lower viewing frame may include only a toggle button to switch between the two displays of ‘CURRENT’ readings (FIG. 3A) and ‘SUGGESTED’ readings (FIG. 3B).

Reference is now made to FIG. 3C showing a representation of a combined screen display 300C, possibly for a centrally managed monitoring system, indicating current orientation and pressure distribution of the pressure-detection surface sections and a possible recommendation plan scheduled on a 24 hour clock basis, for example.

It may be noted that by way of example, the indications refer to three sections of an adjustable pressure distribution surface, supporting head 306, body 308 and feet 310 sections such as a bed in a hospital or a wheelchair.

The screen display 300C may include a display frame 302C with an upper viewing frame, middle viewing frame and lower viewing frame, for example, enabling various selections of display. The upper viewing frame may display the orientation for each section of the adjustable pressure distribution surface head 306, body 308 and feet 310 sections such as a bed in a hospital or a wheelchair. The middle viewing frame may display the calculated recommendation plan over a 24 hour clock for each surface section, enabling one to view all recommendations, or at a specified time period. The lower viewing frame may display the current pressure distribution map for each section, as described hereinabove.

Accordingly, the upper viewing frame of the screen display 300C may provide an indication of the orientations of each monitored section of the subject's body. For example, a user interface may indicate that the head portion is orientated at an angle of +20 degrees to the horizontal, the body portion is orientated at an angle of 0 degrees to the horizontal, and the lower limbs or feet portion is orientated at an angle of +15 degrees to the horizontal. Such an indication may be presented as a text string or may be displayed graphically as a map or an icon or as may otherwise occur to those skilled in the art.

Optionally or additionally, the display frame 302C may include an indicator of the type of display, which may be ‘CURRENT’ readings, for example, to indicate the display is of current readings (as illustrated in FIG. 3A) or ‘SUGGESTED’ readings indicating display of a recommended plan for repositioning or re-orienting (as illustrated in FIG. 3B) and a toggle button to switch between the views.

Optionally or additionally, the screen display 300C may include a control panel 304C, displaying associated information such as the bed number and ‘PREY’ and ‘NEXT’ buttons to move between the beds being centrally managed and monitored.

Optionally or additionally, the screen display 300C may include a bed selector control 324C, displaying a list of currently monitored beds, with the additional ability to select a bed and double click, for example, on the selected item to view its current settings and recommended orientation plan.

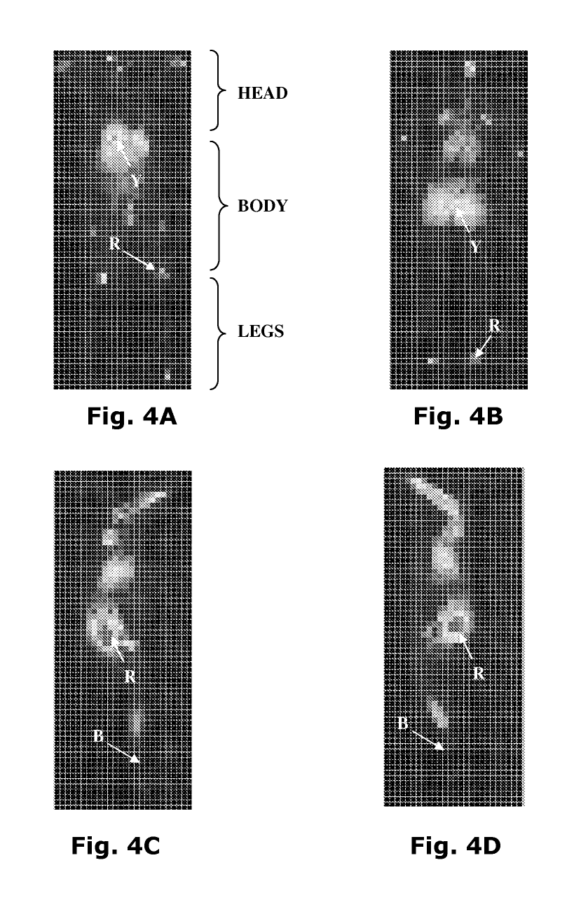

Reference is now made to the pressure maps of FIGS. 4A-D, showing various representations of how pressure data may be displayed on a screen of a display system 170 (FIG. 1A) for an adjustable single section bed. Respectively, FIGS. 4A-D show the pressure distribution for a subject lying on his abdomen (FIG. 4A), his back (FIG. 4B), his left side (FIG. 4C) and his right side (FIG. 4D).

The display system 170 may be a computer in communication with the data storage unit 160, for example. Each display screen shows a matrix of pixels, each pixel representing one sensor of the pressure-detection sheet. The pressure detected by each pixel is represented by a visual indication. A grayscale may be used such that higher pressures are indicated by different shades, darker grays, for example. Alternatively or additionally, colors may be used, for example, indicating high pressure formed between a subject's body and the surface on which the subject rests by displaying the pixel in a distinctive color, such as red (marked with R). Likewise, pixels representing sensors which detect low pressure or no pressure at all may be presented in other colors such as yellow (marked with Y), blue (marked with B) or black. It is understood that other colors or combinations are contemplated for the display screen 170. Furthermore, the ability to customize the pressure scale displayed is contemplated, such as for allowing pressure readings to be scaled up or down depending on the surface the subject is lying on. Such a feature may be useful, for example, to ensure that a caregiver is still alerted to body areas experiencing relatively high pressure even when the patient is lying on an airbed that lowers absolute pressure.

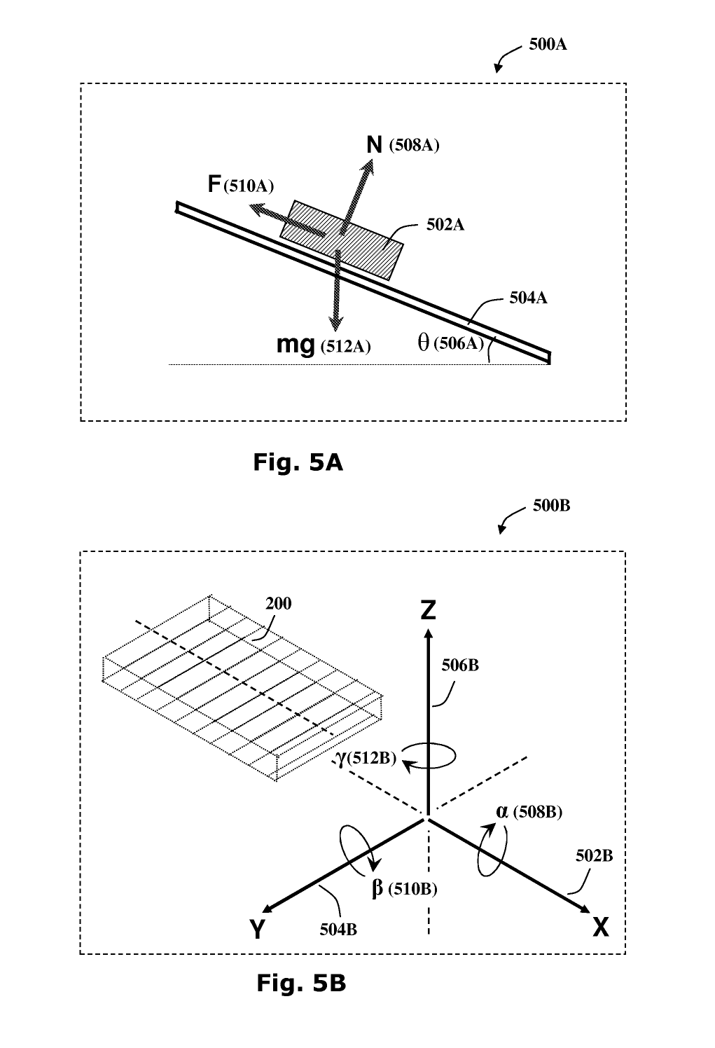

Reference is now made to the force diagram of FIG. 5A showing a two dimensional representation of the system 500A of forces acting when a subject's weight is supported by a tilted surface, such as a section of an adjustable bed including a plurality of sections. The force components system representation 500A includes a section of a subject 502A, having a weight mg (512A), resting on a tilted pressure-detection surface section 504A at an angle θ (506A). The section's weight mg (512A) acts vertically downwards along the Z axis (see FIG. 5B herein below), a normal force N (508A) acts perpendicular to the surface and a shear force F (510A) acts parallel to the surface.

It is noted that some pressure sensor devices measure the force perpendicular to the pressure-detection surface (508A) whereas others may measure the subject's weight mg (512A).

Pressure ulcers may appear in various parts of the body, and their development is affected by a combination of factors, where unrelieved pressure and shearing forces are understood to be principal factors. Knowledge of the interaction of the forces and the resulting stresses (normal and shear stresses) between a subject and a supporting surface may assist caregivers to relieve or reduce external forces, to provide a system for helping to avoid development of pressure wounds.

Shear force may be understood to be the parallel force vector component applied to the subject, and the normal force is the vector component applied in a perpendicular direction, directly against the subject. The shear stress is the shear force per unit area and is measured in Pascals, or Newtons per square meter.

The formula to calculate average shear stress may be given by:

$$τ ( shear stress ) = \frac{F}{A} ( shear force applied / the cross sectional area )$$

where:
τ=the shear stress;
F=the tangential (shear) force applied along the surface;
A=the area of contact (posture contour area) between the subject and the surface material.
   It may be shown by resolving weight (‘mg’) forces that:

N=mg cos θ

F=mg sin θ

leading to the expression:

F=N tan θ

Accordingly, where a pressure sensor measures the normal force N directly, and an alignment sensor measures the tilt angle θ, the shear force F may be calculated.

Reference is now made to FIG. 5B showing an illustration of a three dimensional axis system 500B, identifying various possible angular positioning of a pressure-detection surface section 200, superimposed, for clarity, onto this coordinate system.

The orientation or the angular position of an object, such as a subject lying on an adjustable pressure-detection surface 200 as described herein, refers to how it is placed with reference to each dimension, namely to X, Y and Z. The angle for an axis may be understood as the degree of rotation required to move a subject from a reference orientation to its current placement about that axis. A subject may be tilted longitudinally about the X axis, for example. Additionally or alternatively, rotation of the pressure-detection surface section transversely about the Y axis may result in tilting the subject sideways. Any such orientation change creates a tangential force component with shear stress impact which may cause development of pressure wounds.

The three dimensional axis system representation 500B includes a three dimensional imaginary axis system with an X axis (502B), Y axis (504B) and Z axis (506B) and an indication of the possible rotation angle to change orientation, with a angle (508B) around the X axis, β angle (510B) around the Y axis, and γ angle (512B) around the Z axis. The system also includes a superimposed pressure-detection surface section 200 to clarify orientation changes.

It is noted that the disclosure described herein may use a one dimensional analysis, two dimensional analysis or three dimensional analysis for detecting the current surface orientation, analyzing the required orientation changes and displaying the recommended plan to reduce the possibility of developing pressure wounds.

It is further noted that the analysis may depend on the type of dimensional orientation measurement sensors applied to the pressure-detection surface section. A three dimensional analysis, for example, may only be applicable if an orientation sensor such as a three dimensional accelerometer is part of the measuring system.

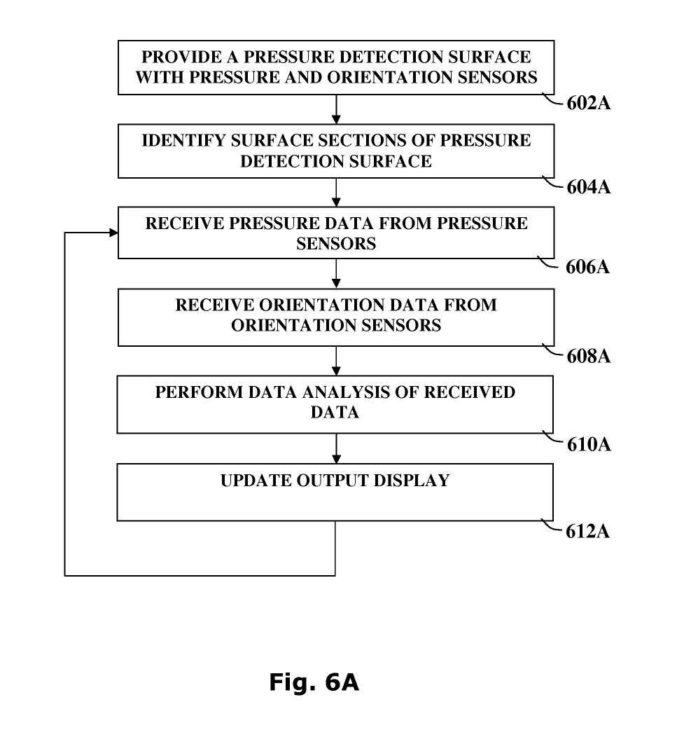

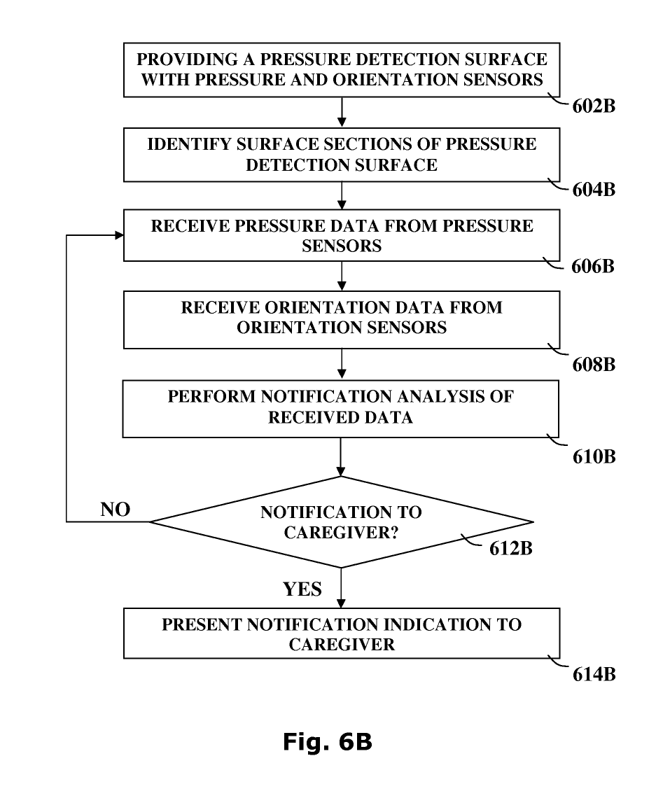

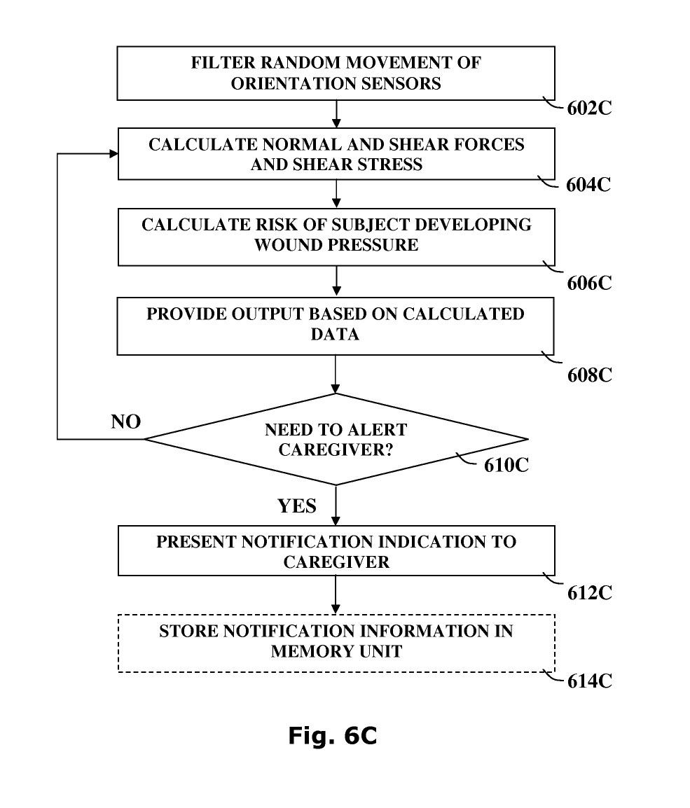

Referring to the flowchart of FIG. 6A, selected actions are indicated of a method for performing pressure and orientation data analysis for the pressure-detection monitoring system. Data analysis may be performed by the processor 150 and updating the local display unit 170, displaying analysis output on a local display of a bed 170B, for example, or sending the analysis output for display on a centralized display unit 370 of the pressure-detection system. The processor 150 may identify different body sections, such as head, body, and foot sections of the pressure-detection surface, and perform analysis separately for each surface section. Thereafter, the data analysis results may be displayed as shown in the illustrative display of FIGS. 4A, 4B and 4C. The identification of the sections may allow pressure distribution to be more easily determined when a patient is in an unusual body position.

It is noted that the processor 150 may be configured to perform the data analysis separately for each section of the adjustable pressure-detection surface. Optionally, contributions of adjacent sections to risk may be combined as required.

It is further noted that recorded data may be logged and stored locally or centrally in data storage 160, including storage of the data analysis results for further analysis and as backup.

According to the method, a pressure-detection surface is provided with a plurality of adjustable sections, equipped with a plurality of pressure sensors and at least one orientation sensor for each adjustable section for measuring pressures and orientations—step 602A. Each section of the pressure-detection surface may be identified—step 604A, pressure data may be received from the plurality of pressure sensors of each surface section of the pressure-detection surface—step 606A, and orientation data may be received from the orientation sensor, an accelerometer, for example, of each surface section of the pressure-detection surface—step 608A. The received pressure data and orientation data may be further analyzed—step 610A and accordingly, the local display unit such as 170 or the central display unit 370 may be updated—step 612A.

Referring to the flowchart of FIG. 6B, selected actions are indicated of a method for generating and presenting a notification alert to a care giver. Data analysis may be performed by the processor 150 and when required an alert may be sent to an output such as the local display unit 170, displaying information on a local display of a bed 170B, for example, or to a centralized display unit 370 of the pressure-detection system. The processor 150 may identify different body sections, such as head, body, and foot sections of the pressure-detection surface, and perform analysis separately for each surface section. Thereafter, the data analysis results may be displayed as shown in the illustrative display of FIGS. 3A, 3B and 3C. The identification of the sections may allow pressure distribution to be more easily determined when a patient is in an unusual body position.

According to the method, a pressure-detection surface is provided with a plurality of adjustable sections, and equipped with a plurality of pressure sensors and at least one orientation sensor, such as an accelerometer or the like for each adjustable section for measuring pressures and orientations—step 602B. Initially, each section of the pressure-detection surface may be identified—step 604B, pressure data may be received from the plurality of pressure sensors of each surface section of the pressure-detection surface—step 606B, and orientation data may be received from the orientation sensor, an accelerometer, for example, of each surface section of the pressure-detection surface—step 608B.

A notification analysis, based on the received data of pressure and orientation, may be performed—step 610B, to define the risk level of developing pressure wounds. If the calculated risk level exceeds a predefined level indicating the need of intervention of a caregiver—step 612B, then an alert may be sent, and the calculated results of the analysis and the recommendations for re-positioning and re-orientation are presented on the display unit—step 614B.

If the analysis results indicate no need for any repositioning, with the current risk level within the acceptable risk range, then the analysis repeats with the new data received from the pressure and orientation sensors.

Referring to the flowchart of FIG. 6C, selected actions are indicated of a method for analyzing received data from the pressure sensors and orientation sensors of each specific section of the adjustable pressure-detection surface to define the risk level of developing pressure wounds, and accordingly sending an alert to a caregiver and presenting the results of the calculated analysis performed by the processor 150 on the local display unit 170, displaying information on a local display 170B of a bed, for example, or at a centralized display unit 370 of the system.

According to the method, the initial step of the notification analysis is to filter the measurements due to the random movement of orientation sensors—step 602C, thereafter the shear force and the normal forces acting on the subject lying over the pressure-detection surface are calculated, and optionally, further calculating the value of the shear stress applied to the subject—step 604C.

The risk level of developing pressure wounds by the subject may be calculated based on the resolved force components and shear stress—step 606C, providing the necessary output for determining the need for sending an indication to the caregiver accompanied with display data for display onto the local display unit 170 or the central display unit 370—step 608C.

The calculated risk level is tested to verify if an alert needs to be sent to the caregiver—step 610C. If the testing indicates an alert is needed, the caregiver is provided with an appropriate notification and accordingly the data results are sent to the display units, mentioned hereinabove, for display and handled by the caregiver—step 612C.

Optionally, the sensor data and the resulting calculated data may be stored in memory or data storage 160, such as for additional analysis and as a reference—step 614C.

### Pressure Mapping System for Wheelchair:

A pressure mapping system for a wheelchair may allow continuous real-time access to pressure data using a pressure sensing cover that may be secured to a wheelchair cushion and back seat and left in place at all times. Such pressure data may be presented on an individual handheld device, allowing caregivers and patients to identify high risk pressure areas and off-load them accordingly, preventing pressure wounds from developing in a seated subject.

A wheelchair may be equipped with a hardware module and a software module, operable to combine a configurable pressure-detection monitoring system.

### The Software Module:

The software module may use a development platform, such as Embedded Linux or the like for its server side, possibly configured to use development environments such as WINDOWS or the like. The user interface may be connectable through a local application or may use a web interface as required.

It may be noted that the server side of the software module may allow the capture of pressure data, and may store the data locally or remotely, perform communications tasks, data analysis, send notifications and messages and communicate with the user to enable monitoring of pressure distribution maps and the like.

It may further be noted that the client side of the software module may support a user interface allowing the configuration of system settings, notification of a weight shift schedule and recommendations, daily target achievements, live and historical images of pressure distributions and the like.

### The Hardware Module:

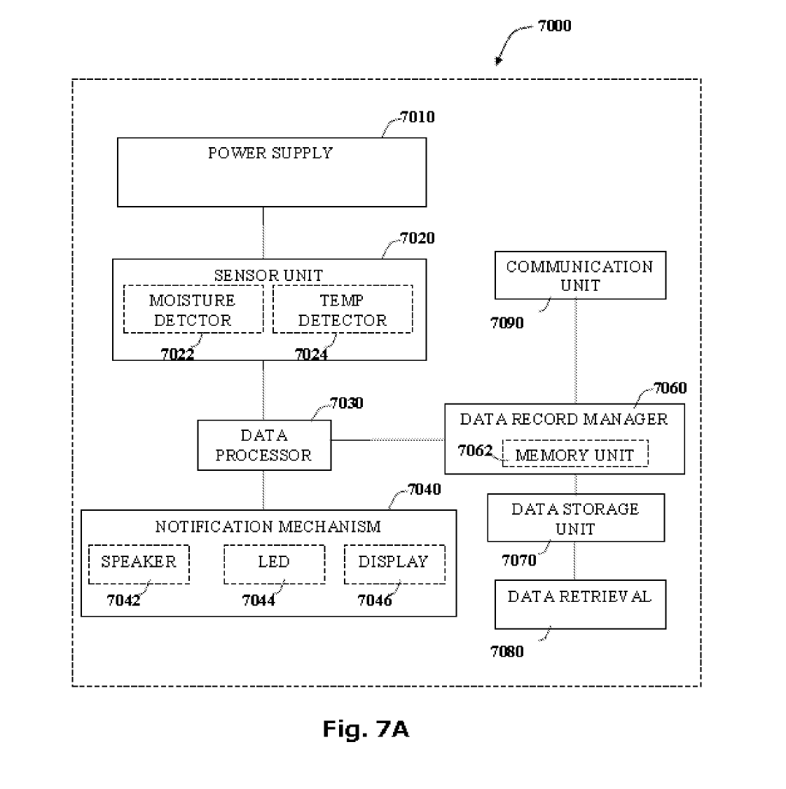

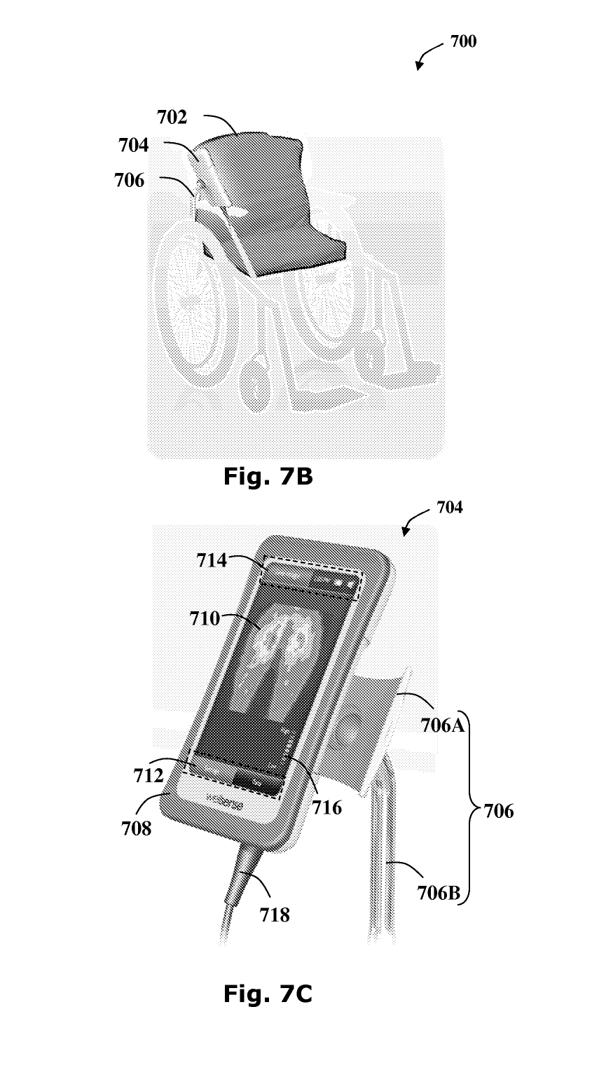

Referring to the block diagram of FIG. 7A, selected elements are presented of a hardware system 7000 for use in a pressure mapping system possibly for integration onto a wheelchair or the like. The hardware module 7000 may include any of the components such as data processor 7030, communication unit 7090, for example using a protocol such as WIFI, BLUETOOTH, ZIGBEE and the like, a data storage unit 7070, a medical grade power supply 7010, a notification unit 7040 including, for example a LED indication 7044, a speaker 7020 to support audible notifications a display 7046 possibly, an LCD display unit supporting touch technology or the like, a power unit, possibly including a rechargeable battery, a sensor unit 7020, for example including a moisture detection unit 7022, and a temperature detection unit 7024 and LCD display unit supporting touch technology.

Optionally, the communication unit may support WIFI 802.11b/g/n with FCC compliance. The unit may further be used as an optional method for data back up and as a service platform providing maintenance and upgrade functionality.

Optionally, the data storage unit may provide internal and external data storage functionality for the purposes of data retrieval and analysis. Where appropriate, the communication with the data storage unit may be encrypted.

Optionally, the battery may support systems' operation for say, 6 hours or so, mainly for the standard/non-powered wheelchair, with a possible indication of the battery status through the user interface.

Optionally, the internal speaker may support audible notifications such as “time for a weight shift” and the like. The notification sound may be selectable and volume may be adjustable via the user interface.

Optionally, the LED indication may provide user indication of the status of the system, such as on/standby/dim/and the like.

Optionally, the moisture detection unit may be configured to alert the patient and caregiver when a predefined threshold has been exceeded, supporting adequate microclimate conditions.

Optionally, the temperature detection unit may be configured to alert the patient and caregiver when a predefined threshold has been exceeded, supporting adequate microclimate conditions.

Furthermore, the moisture detection unit and temperature detection unit may be operable to provide inputs into an algorithm for determining a weight shift schedule.

### Functionalities—Data Storing, Backup and Security:

Where appropriate, the pressure mapping system may further include a data record manager 7060 for supporting data storage, data backup and data security for aspects of data management, while storing collected data on the system's designated database or memory unit 7062. Data records may be stored in at least one data storage unit 7070 according to a recordation schedule.

The data storage main medium may be a Flash memory. A data retrieval mechanism 7080 may be provided via which the data can be gathered from the system, wherein data retrieval may be via either designated SD card or via a wireless module. Such data retrieval mechanisms may require an authentication process with the system. When the wireless module is in use, communications between the system and the web application may be based on TCP sockets or the like.

Optionally, the stored data on the system may be encrypted and may not be accessible without a specific medium.

Where appropriate, all information collected and presented may be compliant with the HIPAA (Health Information Portability and Accountability Act) standard.

Optionally, when the system is collecting data, the database may be in the form of a table, for example. Variously, the set of events that may be recorded and stored in the database file may be selected from events such as: user personal settings to enable matching the system and data to a specific user for analysis purposes; weight shift notification appearance; weight shift completion; partial weight shifts that may be used for movement detection; system shut down; system powering up; and scheduling or setting of modifications. Optionally, all database data records may further include a timestamp field representing the time of data entry occurrence.

Where appropriate, additional events such as user interactions, new software and hardware features, malfunctioning of the hardware or software modules and the like, may be added, accordingly.

Where appropriate, the mapping system may periodically capture pressure measurements and capacitive data generated by the pressure exerted over the surface. Those measurements may be the ones translated into the pressure map in the monitoring stage and are the same ones that eventually will be viewed in the data analytics tool via a player such as described herein. All data may be protected behind an approved firewall.

It is noted that data analysis and reporting functionalities may use an encrypted web application designed to support various operating systems. Where required, the data retrieved from each system may be displayed on a designated secured/encrypted web application which is installed on facility equipment; hence it will be secured as another piece of equipment in the facility.

It is further noted that accessing the data of the mapping system through a web application may allow the authorized user to manage all aspects of data analysis.

Integration with Health Information Networks:

Where appropriate, the architecture of the mapping system may be operable to integrate with the health information networks based on a communication platform between the web application and the network protocols. In particular, the system may be operable to integrate with the Veterans Health Information Systems and Technology Architecture (VistA), the Computerized Patient Record System, (CPRS) or other client—server interface that allowing health care providers to review and update a patient's electronic medical records.

Optionally, the scope of information to be included and presented in the health information network such as VistA, for example, may include various parameters such as, inter alia: user personal information and settings, schedules of weight shifts completion, snapshots of positions throughout a selected period of time such as hourly, daily, monthly and the like, achievement of personally configured targets of weight shifts and the like as well as combinations thereof. Additionally or alternatively, systems may integrate information including user triggered events, new software, hardware functionalities and the like.

### Embodiments of a Wheelchair Pressure Mapping System:

Reference is now made to the illustration of FIG. 7B, showing a wheelchair based embodiment that may be used to monitor pressure exerted by a seated subject, configured for using a continuous pressure mapping system 700, which may be referred to as ‘MAP for wheelchair’. The continuous pressure mapping system 700 may allow for a pressure sensing cover to be secured to the wheelchair cushion and back seat and left in place at all times so that caregivers and patients have real-time pressure data presented on an individual handheld device to identify high risk pressure areas and off-load them accordingly.

The continuous pressure mapping system 700 may include a textile pressure-sensing cover 702 and a monitor 704 attached to a mounting system 706. The textile pressure-sensing cover 702 may incorporate an array of pressure sensors as described above. The system measures the pressure level in each sensor, calculating the pressure distribution in real time. The capacitive measurement technology is highly accurate and provides a live image of the pressure distribution over the seating and the back support areas. The pressure sensing cover 702 is designed for uninterrupted monitoring and may be comprised of biocompatible external fabric, for example.

The continuous pressure mapping system 700 may further include an optional data collection module which may gather data according to users' requirements. Patients and caregivers may review patterns of weight-shifts and pressure redistribution over time to adapt treatment plans and evaluate current seating systems over the entire period that a patient is in a wheelchair.

It is noted that the pressure mapping system 700 may guide patients and caregivers through accurate and timely weight shifting, while providing relevant notifications when weight shifts becomes necessary based on predefined schedules and positions.

It is further noted that pressure mapping system 700 may allow estimation of areas of pressure that could represent risk over time and the system may produce reports and support the management of patient information related to effective repositioning.

Reference is now made to the illustration of FIG. 7C, showing a monitor 704 for monitoring the continuous pressure mapping system 700. The monitor 704 may be located on a mounting system 706 may have a touch-screen interface using a user-friendly, high resolution display that may graphically indicate the range of high to low pressure readings with any change in position or weight shift. Accordingly the monitor 704 may provide appropriate alerts and notifications.

The monitor 704 may be mounted on a mounting system 706 having an attachable tilted unit 706A and a supporting leg 706B and may include a monitor casing 708, a monitor display screen 710, monitor control buttons 712, a monitor status display 714, a display resolution scale 716 and a connectible cable 718.

The monitor display screen 710 may use a touch-screen interface, or the like, supporting interactivity and simple and approachable for both the wheelchair users and their caregivers. The touch operated user interface may allow personalization of the system to match the unique weight shift requirements of each user.

Optionally, the monitor control buttons 712 may be used to handle setting of the monitor and data management, for example. A setting button may enable one to configure personal attributes of a patient, define a weight shift schedule, set a target, set notification types and the like.

Optionally, the monitor status display 714 may be used to provide an indication of the type of display, such as “Live Image”, presenting a pressure distribution of the currently seated subject, status of the rechargeable battery, volume control, date and time and the like.

Optionally, the display resolution scale 716 may provide a resolution indication related to the display, for example having colors indicating different pressure distributions.

Optionally, the connectible cable 718 may be operable to provide operational power or rechargeable power for the monitor rechargeable battery and network connectivity.

It is noted that the current design of the monitor 704 is presented in a non-limiting manner by way of example, so as to better illustrate the utility of the system. Additionally or alternatively, other parameters may be introduced related to settings and monitoring, and may change, add or remove display scenarios, and the like.

It is further noted that the software required for monitoring and communication purposes of the may use a development platform such as Embedded Linux. Additionally and alternatively the system may use a WINDOWS development platform or the like.

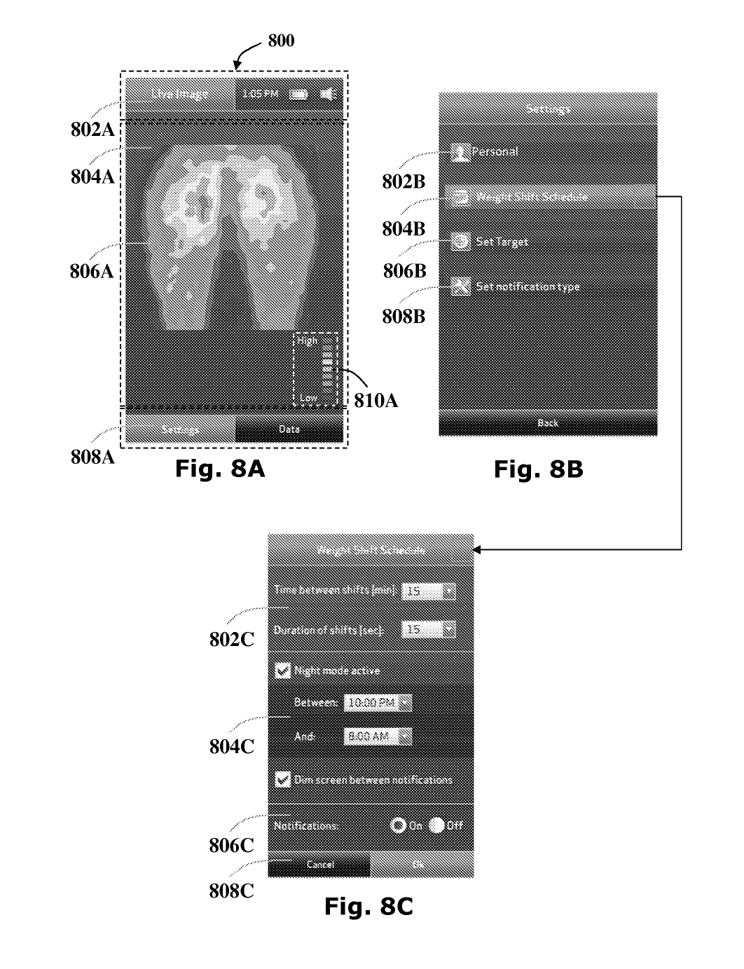

Reference is now made to the illustration of FIGS. 8A-C, showing a monitor display 800 and functionality settings of the continuous pressure mapping system 700 (of FIG. 7A). The monitor display 800 shows the possible functionalities of configuring the mapping system such as configuring personal setting, notifications, target achievements, and specifically the configuration of the ‘Weight Shift Schedule’, as an example.

FIG. 8A shows a possible live image screen display, coupled with possibly the setting of system functionalities.

The main screen 804A shows a live image display 806A, representing a pressure distribution map of the seat and back areas of a seated subject. The live image display 806A changes at every movement to indicate the real time pressure distribution created on the surface by the seated subject.

The bar on the top 802A presents the status of the audible signal that may be set to ‘on’ or mute and the battery charging status.

It is noted that the continuous pressure mapping system 700 (of FIG. 7A) may be able to detect any movement, including weight shifts and to record them. If a user is out of the chair, the system 700 (of FIG. 7A) may have the capability to pause the monitoring and resume upon a user's return. Optionally, events may be detected, such as a patient falling or indications that the patient may potentially fall from the chair. Accordingly an alert may be set so as to notify a caregiver.

Tabs 808A available may include ‘Settings’ configured to manage the pressure detection and mapping system settings and ‘Data’ configured to manage system data, as shown in the illustrative display of FIGS. 8B-C and as described hereinafter.

The pressure indication color index 810A may provide a scale, say from red, red-orange, orange, yellow, yellow-green, green, green-blue, light blue to blue. The scale may allow various levels of pressure distribution to be indicated, for example from high level (red) to low level (blue). FIG. 8B shows a ‘settings’ screen enabling the pressure mapping system configuration and settings of the system's functionalities for the desired operability for a patient.

Where appropriate, the ‘Settings’ screen may allow the patient or caregiver to create a set of personal settings to accommodate the individual needs of each patient, while running the pressure mapping system.

Where appropriate, the ‘Personal’ button 802B may enable inserting the patient's personal data for analysis and tracking.

Where appropriate, the ‘Weight shift schedule’ button 804B may enable setting of the shift schedule, described hereinafter for FIG. 8C.

Where appropriate, using the ‘Set a Target’ button 806B, the caregiver or the user may set a daily target. In this mode of operation, the patients may be presented with their own personal weight shifts accomplishments throughout the day and will be credited for their success meeting the target.

The ‘Set notification type’ button 808B may enable the patient or caregiver to select a preferable tone for audible notifications.

FIG. 8C shows a ‘Weight Shift Schedule’ screen which may enable configuration of the setting of the patient shifting schedule for the pressure mapping system.

Where appropriate, the ‘Weight Shift Schedule’ display may enable the patient or caregiver to set a time interval for a ‘Weight Shift Schedule’ field 802C, configuring the interval time between weight shifts and setting the duration of each weight shift.

Where appropriate, the ‘Weight Shift Schedule’ display may enable the patient or caregiver to configure the “Night mode active” and “Dim screen between notifications” fields 804C. Optionally, when night mode is activated then notification will stop during the time frame set.

Where appropriate, the ‘Notifications’ field 806C may provide the patient or caregiver with the option to decide if the audible signal for the weight shift time is enabled or disabled.

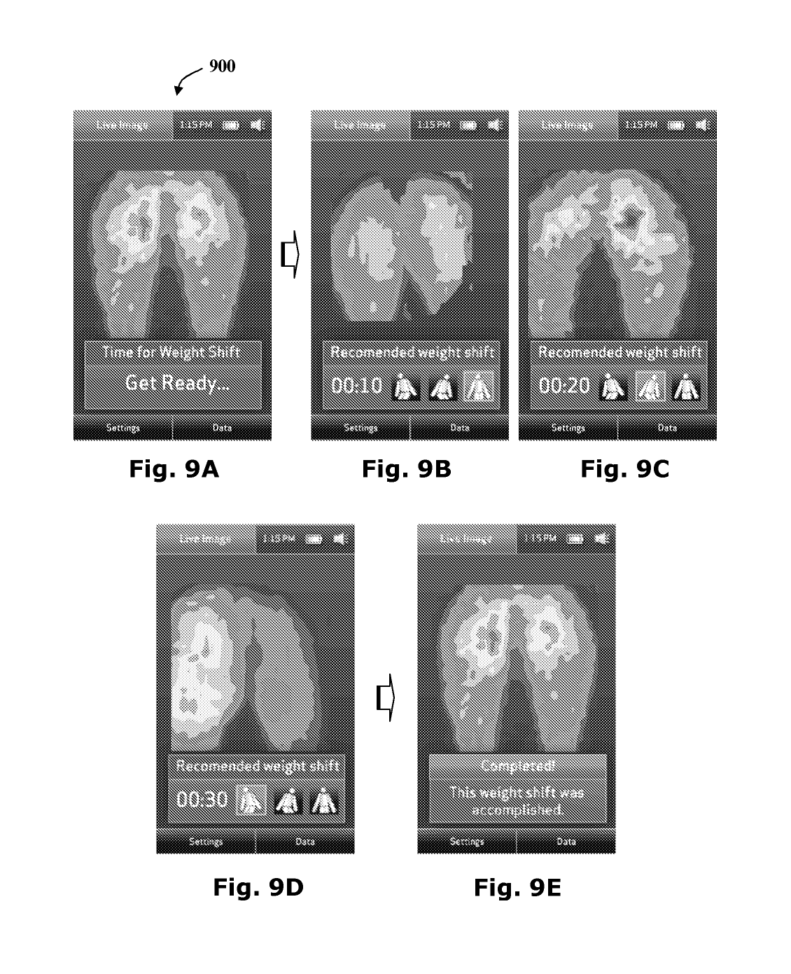

Reference is now made to the illustrations of FIGS. 9A-E showing a monitor display 900 of performance of the functionality settings of the ‘Weight Shift Schedule’.

Where appropriate, when the weight shift schedule has been configured (as described hereinabove, FIGS. 8A-C), the pressure mapping system will notify the patient/caregiver when the time to shift has arrived. Optionally, visual and audible notifications are available based upon configuring the pressure mapping system.

FIG. 9A represents a possible screen showing notification alerts, when time for the next weight shift arrives.

It is noted that all alerts cease when no pressure/weight is detected on the surface (textile pressure-sensing cover 702 of FIG. 7A) of the pressure mapping system and/or when the weight shift has been accomplished.

FIGS. 9B-D represent possible screens of the personal settings configuration, where the patient/caregivers may select the recommended weight shift position the patient should follow. This selection may be based on the patient's ability to move, for example, and a personal selection may be made from of a list of possible positions. Based on the selection made, the pressure mapping system may provide a recommendation to redistribute weight by shifting position. The counter may indicate the time left for each position.

FIG. 9E represents a possible screen showing a patient's notification when the weight shift has been accomplished. The pressure mapping system is able to automatically detect and record weight shift/partial movements on the surface (textile pressure-sensing cover 702 of FIG. 7A).

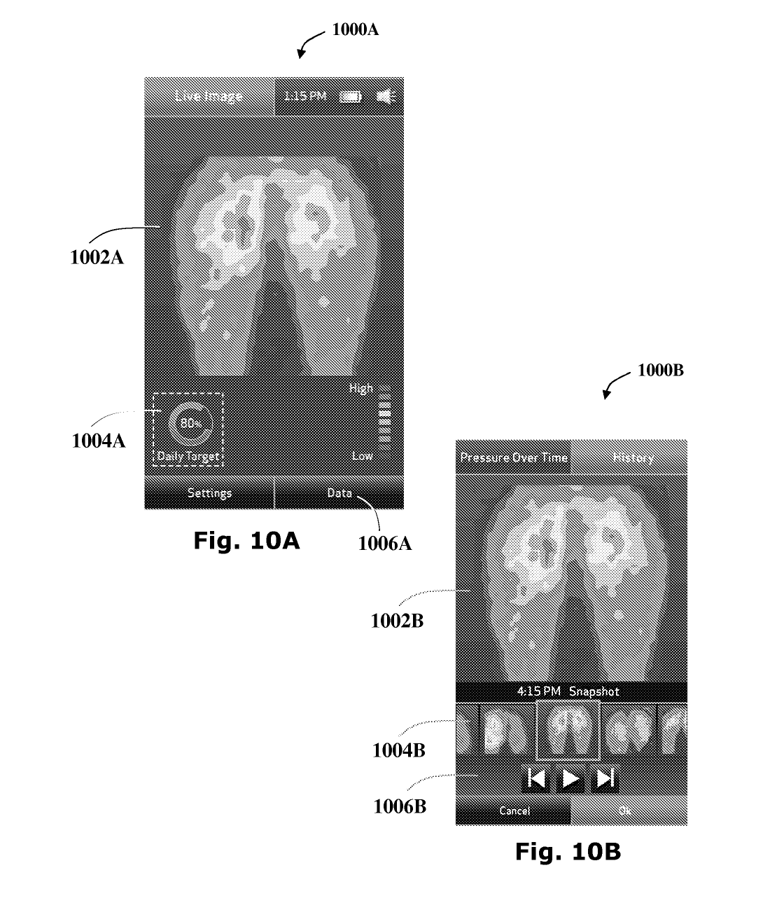

Reference is now made to the illustration of FIG. 10A, showing a monitor display 1000A configured to manage a daily target achievement plan. The daily target achievement plan may be configured by the caregiver or the patient to reach an individual target for weight shifts to be accomplished daily, preventing pressure wounds from developing to a seated subject.

The monitor screen display 1000A may include a pressure distribution image section 1002A, a daily target indicator 1004A and an access button bar 1006A.

Accordingly, the pressure distribution image section 1002A may display a live image of the currently selectable pressure distribution image and the daily target indicator 1004A provides an indication of the current progress level of the daily preconfigured achievement plan. Further, the access button bar 1006A provides optional menu selection to access the ‘data’ configuration screen or ‘setting’ configuration screen.

Where appropriate, a patient or a caregiver may set an individual target program of weight shifts for the seated subject, to be accomplished as a daily routine. A progress bar may indicate how close/far the patient is from reaching the preset daily goal, as may be indicated in a percentage level, for example, by the daily target indicator 1004A.

The target setting menu may take into account the main three parameters as a weighted average: time between shifts, duration of each shift and the night mode settings. This may present a sophisticated feature that may calculate a maximum number of allowed shifts in a predefined period of time to avoid users ‘tricking’ the system, for example by performing many weight shifts in a short period of time to meet the set target.

It is noted that the target setting menu is accessible through the menu option of ‘Set Target’ as specified in FIG. 8B, 806B.

It is further noted that the night mode setting is accessible through the menu option of ‘Weight Shift Schedule’, checking the option ‘Night mode active’ as specified in FIG. 8B, 804B and reflected in FIG. 8C, in section 804C.

Reference is now made to the illustration of FIG. 10B, showing a monitor display screen 1000B configured to display previously captured pressure map images. The system may be configured to capture pressure distribution data periodically throughout the day. The captured images may further be processed, analyzed, and displayed accordingly.

The monitor screen display 1000B may include a pressure distribution image section 1002B, a snapshot selection list 1004B and a snapshot history scroll bar 1006B.

Accordingly, the pressure distribution image section 1002B may display a snapshot image of the current pressure distribution image of the selected history event and the snapshot selection list 1004B provides a selectable list presented in an iconized form, enabling one to select the desired event to process or review. Further, the snapshot history scroll bar 1006B allows scrolling through the list of existing snapshots of the various events stored.

The pressure mapping system may be operable to capture pressure measurements throughout the day. Such capturing of pressure measurements may be streamed, performed throughout the day or triggered manually to capture a pressure map image at a point of interest or reflecting an event.

It may be noted that the streamed pressure images may be captured constantly throughout the day, or may be configured to be performed periodically, starting at a given time, for a duration and continue every time interval, for example.

Viewing previously captured pressure map images may be accessible through selecting the data tab, optionally providing two presentation modes: ‘Pressure over time’ and ‘Snapshot capture’.

The view of ‘Pressure over time’ allows historical viewing of displays of accumulated pressure points ‘streamed’ throughout the day. Such streamed images may help the patient or the caregiver to identify high risk pressure areas and to off-load them accordingly. The pressure-over-time indication may also be valuable for cushion evaluation/adjustment and overall repositioning on the wheelchair.

The view ‘Snapshots’ may allow the patient or caregiver to select the viewing of one or more captured pressure map images from the set that were manually saved in the past 24 hours, periodically to reflect a specific event or point of interest.

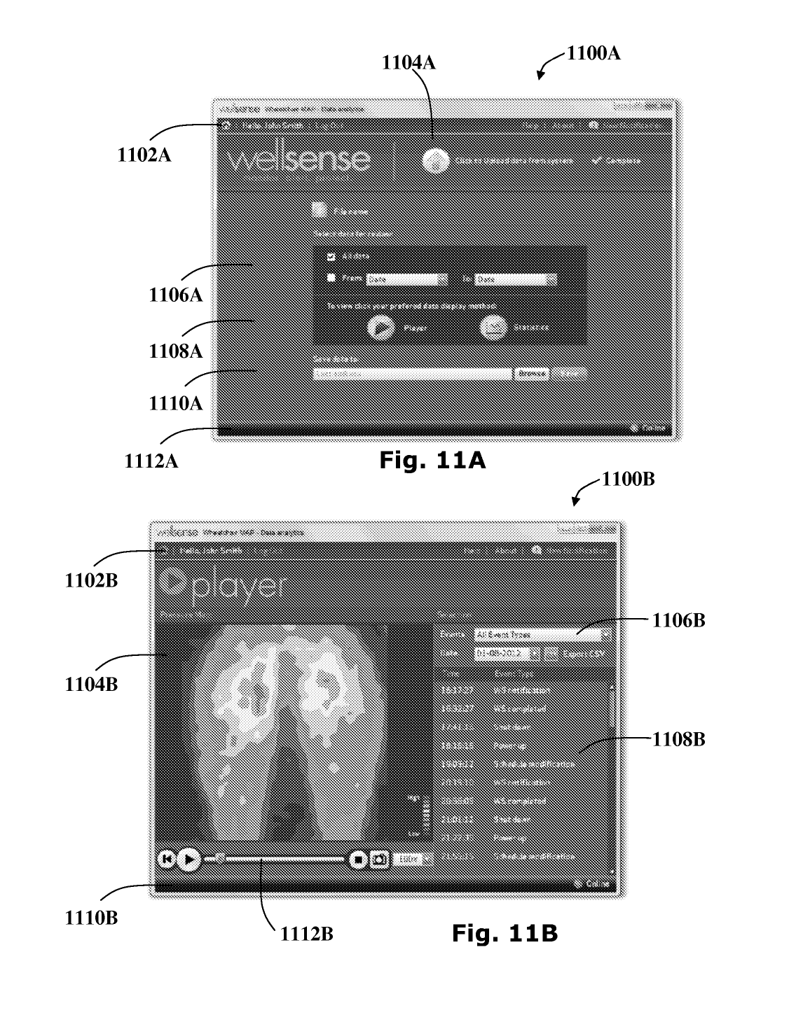

Reference is now made to the illustrations of FIGS. 11A-B showing web application screenshots 1100A and 1100B for data analysis purposes.

It may be noted that the web application may allow the authorized user to perform various management tasks related to data analysis and processing. Additionally or alternatively, the web application connectable to the mapping system may provide for configuring setting and entry requirements for data analysis.

FIG. 11A provides a possible entry screen of the mapping system for a wheelchair web application, showing various possible data related tasks. The entry screen display 1100A may include a login section 1102A, an upload data section 1104A, a date selection section 1106A, a data review and analysis format section 1108A, a saving data section 1110A and a status bar 1112A.

The login section 1102A provides for users' management. Users may be authorized according to the Veterans Affairs protocols or the like. This may ensure a high level of data security and confidentiality. It is noted that only certified users may be able to log in to manage and interact with the application, based on the users' level of authorization. Accordingly, some users may have the permission of viewing only, while others may be allowed to modify, perform analysis, change configurations and the like. It is further noted that the upload data section 1104A may allow uploading of data from a selected pressure mapping system, using some retrieval mechanism, for example removal of an SD card or a wireless communication system. The date selection section 1106A may allow selecting the dates for data review and analysis.

The data review and analysis formats section 1108A may allow data to be reviewed and analyzed in a statistical format such as graphs, charts and the like or via a player mode of conceptual presentation as described hereafter.

The save data section 1110A may allow for uploading files on a PC or the facility server. Saving the files may allow for further integration of the data with a health information network such as the VistA information system or the like. Data may be stored and saved for historical recording and patient weight shifts tracking.

FIG. 11B provides a possible player analysis screen of the mapping system for a wheelchair web application, showing various screen sections. The player analysis tool may enable selection of an event from the event list to be played for further processing or analysis purposes.

The player analysis tool screen display 1100B may include a login status section 1102B, a pressure map display section 1104B, an event type selection section 1106B, an event list section 1108B,a status bar 1110B and a playing button 1112B.

The login status section 1102B may provide for users' current logged in status, logout button, user name, and indication for new notifications and may offer access to a help section of features. The pressure map display section 1104B may provide a portion of the screen to display the streamed video content of a selected event, which may be a series of pressure distribution maps representing the captured data sequentially. Further, the event type selection section 1106B may allow for selecting the dates for data processing and review or further data analysis, while the status bar 1110B may allow showing whether the system is currently online. The playing button section 1112B may allow various activation options while “streaming” video content of selected events, which may involve the sequential presentation of a series of pressure distribution maps representing the selected event.

The player analysis tool allows a “video streaming” mode for displaying the pressure distribution maps representing the selected event on a specific selected date, for a specific mapping system and a specific user.

Additionally or alternatively, snapshots of pressure distribution may be configured to be captured, manually or periodically, and saved for further analysis at a later stage. Optionally, any captured distribution map image may be viewed at any time and may further be exported into a CSV (Comma-Separated-Value) file for further analysis.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number, and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described hereinabove as well as variations and modifications thereof, which would occur to persons skilled in the art upon reading the foregoing description.

1-33. (canceled)

34. A method for processing pressure wound prevention system measurements comprising:
providing a pressure wound prevention system configured to detect pressure and orientation;
providing at least one pressure-detection surface comprising a plurality of pressure sensors configured to detect pressure distribution and at least one orientation sensor configured to detect angle indication;
identifying surface sections of said pressure-detection surface;
receiving pressure data from said pressure sensors for each surface section;
receiving orientation data from said orientation sensor for each surface section; and
presenting a notification indication to a caregiver.

35. The method of claim 34, wherein said presenting a notification to a caregiver comprises:
filtering random movement of said orientation sensors for each surface section;
calculating vertical forces and shear forces of a subject over each said surface section of said pressure-detection surface;
calculating a risk of a subject developing pressure wounds;
providing an output based upon calculated data; and
sending an alert to a caregiver indicating a repositioning requirement.

36. The method of claim 34, further comprising storing said data in at least one data storage unit.

37. The method of claim 34, wherein said providing an output includes displaying a pressure distribution map for each said section on said at least one display unit.

38. The method of claim 34, wherein said providing an output includes displaying a current orientation indication and a suggested orientation indication for each said section on said at least one display unit.

39. The method of claim 38, wherein said orientation indication is three dimensional indicating lateral tilt values.

40. A pressure wound prevention system operable to prompt at least one caregiver to reposition a subject at risk of developing a pressure wound, said system comprising:
at least one pressure-detection surface comprising a plurality of pressure sensors configured to monitor pressure exerted upon said subject;
at least one processor operable to interpret and analyze data from said sensors;
at least one data storage unit operable to store said data from said sensors and said processor;
at least one notification mechanism in communication with said at least one processor, said notification mechanism operable to initiate at least one repositioning alert ;
a power supply unit operable to provide operational power;
a data recording manager in communication with said at least one data storage unit and said at least one processor and operable to manage data collection;
a communication unit operable to provide access to said data; and
at least one display unit configured to present said repositioning alert.

41. The pressure wound prevention system of claim 40, wherein said pressure-detection surface comprises a plurality of adjustable sections.

42. The pressure wound prevention system of claim 40, wherein said data recording manager comprises at least one memory unit configured to store said data from said sensors and said processor according to a recordation schedule.

43. The pressure wound prevention system of claim 40, wherein said notification mechanism is operable initiate a display of a series pressure distribution maps representing said data sequentially.

44. The pressure wound prevention system of claim 40, wherein said pressure-detection surface further comprises at least one orientation sensor configured to monitor an angle of said pressure-detection surface.

45. The pressure wound prevention system of claim 44 wherein said at least one orientation sensor comprises at least one accelerometer.

46. The pressure wound prevention system of claim 44, wherein said at least one orientation sensor comprises at least one three dimensional accelerometer.

47. A method for processing pressure wound prevention system measurements comprising:
providing a pressure wound prevention system configured to detect pressure;
providing at least one pressure-detection surface comprising a plurality of pressure sensors configured to detect pressure distribution;
identifying surface sections of said pressure-detection surface;
receiving pressure data from said pressure sensors for each surface section;
storing data records in at least one data storage unit according to a recordation schedule; and
presenting a notification indication to a caregiver wherein said notification indication comprises a three dimensional indication of lateral tilt values.

48. The method of claim 47, wherein said presenting a notification to a caregiver comprises the steps of:
calculating vertical forces and shear forces of a subject over each said surface section of said pressure-detection surface;
determining a risk of a subject developing pressure wounds;
providing an output; and
sending an alert to a caregiver indicating a repositioning requirement.

49. The method of claim 48, wherein said providing an output comprises displaying on a display unit a series of pressure distribution maps representing said data sequentially at regular time intervals.

50. The method of claim 48, wherein said providing an output comprises displaying on a display unit a recommendation plan of a weight shift schedule for each said section.

51. The method of claim 48, wherein said providing an output comprises generating a daily target achievement plan displayed on at least one display unit.

52. The method of claim 48, wherein said providing an output comprises displaying a current orientation indication and a suggested orientation indication for each said section on at least one display unit.

53. The method of claim 47 further comprising:
receiving from said at least one orientation sensor orientation data for each surface section; and
filtering random movement of said at least one orientation sensor for each surface section.
